(19) United States
(12) Patent Application Publication
(10) Pub. No.: US 2023/0105649 A1
(43) Pub. Date: Apr. 6, 2023

(54) DETERMINING WEATHERING INDICES BY X-RAY DIFFRACTION

(71) Applicant: Saudi Arabian Oil Company, Dhahran (SA)

(72) Inventor: Mohamed Soua, Dhahran (SA)

(21) Appl. No.: 17/494,285

(22) Filed: Oct. 5, 2021

(51) Int. Cl. G01N 23/207; G01N 23/223; G01N 1/28; G01N 23/2005; G01N 33/24

(52) U.S. Cl. CPC G01N 23/207; G01N 23/223; G01N 1/286; G01N 23/2005; G01N 33/24; G01N 2001/2866; G01N 2223/616; G01N 2223/345

(57) ABSTRACT

Methods for determining a weathering index using x-ray diffraction (XRD) data are provided. An exemplary method includes obtaining XRD data of a weathered rock sample, and calculating the weathering index using a formula developed to use the XRD data.

### TECHNICAL FIELD

The present disclosure is directed to the determination of the weathering indices using X-ray diffraction mineralogy.

### BACKGROUND

The location of reservoirs for crude oil and natural gas often depends on the identification of rock types and age in cuttings from wells. A weathering index is a quantitative measure of the extent of weathering of rock. The weathering index, such as the chemical index of alteration (CIA), can be used with other measurements to classify rock types in layers as a well is drilled. Further, it is useful for prediction models to assess the strength and deformational properties of rocks during drilling. In general, it can be indicative of maturity of the provenance sediments, paleoclimate of the source area and paleosol or rip up clasts in fluvial channel facies derived from a paleosol. The chemical weathering of these materials results in the formation of clay minerals.

Chemical weathering indices are commonly used for characterizing weathering profiles by incorporating molar major element oxide chemistry into a single metric for each sample. A number of weathering indices have been developed.

The chemical index of alteration (CIA) was described in Nesbitt, H., & Young, G. M., “Early Proterozoic climates and plate motions inferred from major element chemistry of lutites,” Nature, 299(5885), 715-717 (1982). The CIA is based on the assumption that the dominant process during chemical weathering is the degradation of feldspars and the formation of clay minerals. The CIA is calculated by the formula CIA={Al₂O₃/(Al₂O₃+CaO*+Na₂O+K₂O)}×100. In this formula, and each of those herein, the values represent the mole fraction of the chemical compounds listed versus the total sample.

The chemical index of weathering (CIW) was described in Harnois, L., “The CIW index: a new chemical index of weathering,” Sedimentary Geology, 55(3), 319-322 (1988). The CIW is nearly identical to the CIA, except that it eliminates K₂O from the equation. The CIW is calculated by the formula CIW={Al₂O₃/(Al₂O₃+CaO*+Na₂O)}×100.

The plagioclase index of alteration (PIA) was described in Fedo, C. M., Wayne Nesbitt, H., & Young, G. M., “Unravelling the effects of potassium metasomatism in sedimentary rocks and paleosols, with implications for paleoweathering conditions and provenance,” Geology, 23(10), 921-924 (1995).The PIA is an alternative to the CIW and is calculated by the formula PIA={(Al₂O₃−K₂O)/((Al₂O₃−K₂O)+CaO*+Na₂O)}×100.

The weathering index of Parker for silicate rocks was described in Parker, A., “An index of weathering for silicate rocks,” Geological Magazine, 107(6), 501-504 (1970). The WIP is based on the proportions of the individual mobilities of sodium, potassium, magnesium and calcium, and is calculated by the formula WIP={(2×Na₂O/0.35)+(MgO/0.9)+(2×K₂O/0.25)+(CaO*/0.7)}×100.

In the above formulas, the calcium is based on CaO*, which is exclusively the calcium content incorporated in silicate fraction. Further, in each of the equations above, the chemical formulas represent the fraction of that chemical in the compound. The compounds are generally analyzed by x-ray fluorescence (XRF), as XRF is a faster technique than chemical analysis. However, XRF is generally limited to about 15 samples per day for a single analysis instrument.

### SUMMARY

An embodiment described in examples herein provides a method for determining a weathering index using x-ray diffraction (XRD) data. The method includes obtaining XRD data of a weathered rock sample, and calculating the weathering index using a formula developed to use the XRD data.

Another embodiment described in examples herein provides a method for constructing a weathering index for using x-ray diffraction data by obtaining samples of weathered rock, grinding the samples, preparing a powder mount of each sample, obtaining x-ray fluorescence (XRF) data for each sample, calculating a second weathering index using the XRF data, and constructing the weathering index using the XRD data, based, at least in part, on the second weathering index.

### BRIEF DESCRIPTION OF DRAWINGS

FIG. 1 is a drawing of a wellbore drilled through different subterranean formations to reach a reservoir formation.

FIG. 2 is a process flow diagram of a method for determining formulas for calculating weathering indices using data obtained from x-ray diffraction (XRD).

FIG. 3 is a process flow diagram of a method for calculating weathering indices using formulas generated from x-ray diffraction (XRD) data.

FIG. 4 is a plot of a diffraction pattern of minerals used to model weathering indices.

FIG. 5 is a plot of depth profiles comparing different indices of weathering.

FIG. 6 is another plot showing that depth profiles of the different indices are changing according to the profiles determined by XRF.

FIG. 7 is a plot comparing the chemical index of alteration (CIA) determined by XRF with the CIA determined by x-ray diffraction (XRD).

FIG. 8 is a plot of depth profiles comparing the CIA determined by XRD with the CIA determined by XRF.

FIG. 9 is a plot of depth profiles comparing trends in the CIA determined by XRD with trends determined by XRF.

### DETAILED DESCRIPTION

The importance of the weathering index to determine layer composition during drilling leads to hundreds of cutting and core samples to be analyzed. As mentioned, these are generally used to construct the sub-areal intensity history of a given section. However, given the number of samples that are submitted and limitation on the speed of the XRF analysis, the results may take several hours, which may slow drilling.

Embodiments described in examples herein provide a method for calculating weathering indices from x-ray diffraction (XRD) patterns. These indices model the degradation of feldspars and the formation of other minerals. The use of XRD data to calculate weathering indices is tested on samples from wells drilled in the Unayzah (Permo-Carboniferous) formation of Saudi Arabia. In contrast to the slower XRF technique, an XRD instrument can analyze from about 24 to about 48 samples per day. The method is based on formulas incorporated into a spreadsheet, in which results take a very little time to be produced and computed.

In the method, the molar major element oxide chemistry used by other indices is replaced by determining bulk mineralogy by powder XRD patterns. The powder XRD results indicate that the depth profiles of the CIA, CIW, PIA, and WIP indices, determined by mineralogy, are changing according to the profiles generated by XRF, e.g., the molar major element oxide chemistry.

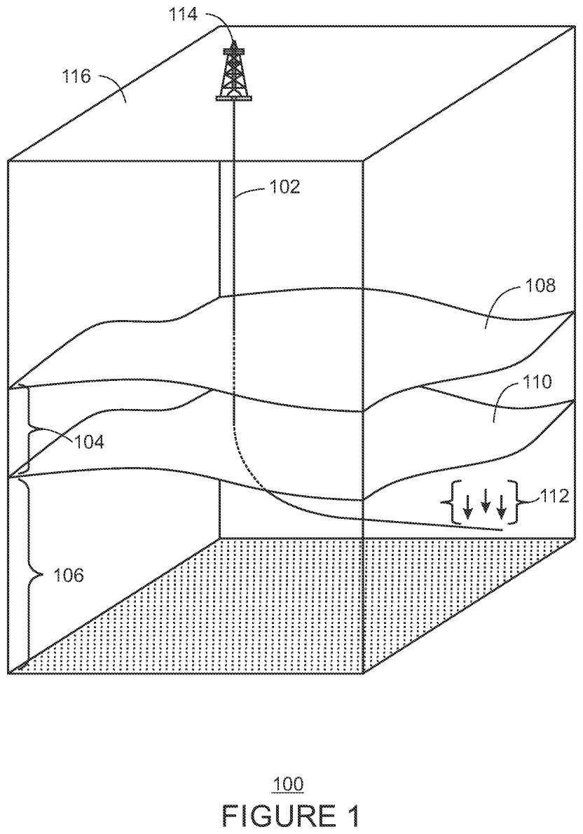

FIG. 1 is a drawing of a wellbore 102 drilled through different subterranean formations 104 to reach a reservoir formation 106. Each of the formations 104 and 106 comprises multiple rock layers, such as layers 108 and 110. As an example, a cap rock layer 110 overlies the reservoir formation 106. Identifying weathering indices for the different layers, such as the layers in the reservoir formation 106 will help to identify the layers, and may help to locate productive locations 112 in reservoir layers 106. This information may be used, for example, in directional drilling of the wellbore 102 from a drilling rig 114 from the surface 116.

Although subterranean layers are discussed as an example of an application, the weathering indices may be used in any number of applications. The results of weathering indices are used by many specialists within earth science, including, for example, sedimentologists, chemostratigraphers, and stratigraphers, among others. Applications may include research applications on the formation of sediments in silt layers, such as in lakes or alluvial fans, or the location of target layers for example, for gold or diamond production from silt layers at the ocean floor. Additional applications may include studies of weathering of buildings, roadways, and other infrastructure elements.

The lower costs and higher throughput of the techniques described herein can increase the utilization of weathering indices, for example, in mapping deposits. XRD analysis, which can provide the mineral amounts used to implement this approach, has lower costs and faster turnaround when compared to XRF or Inductively Coupled Plasma (ICP) technologies. Further, it is widely deployed in oil and gas exploration companies. Conversely, geologists around the world may use the techniques represented by the mineralogical factors in a spreadsheet or an integrated software.

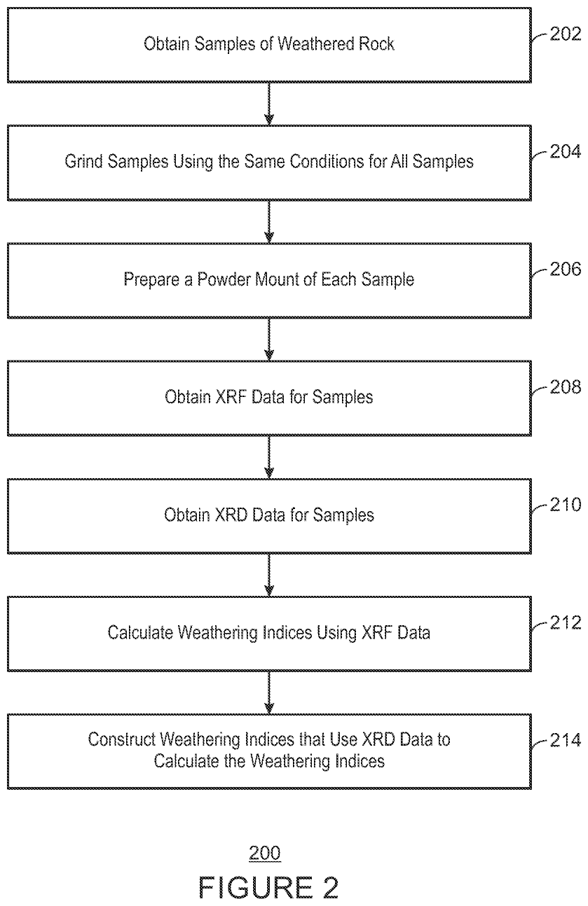

FIG. 2 is a process flow diagram of a method 200 for determining formulas for calculating weathering indices using data obtained from x-ray diffraction (XRD). The method begins at block 202 when samples of weathered rock are obtained. These may be cuttings from a wellbore, rock samples from a surface formation, sediment samples, and the like.

At block 204, the samples are ground using the same preparation techniques for all samples. In some embodiments, a micronizing mill is used to preserve the crystal lattice. This may help to avoid broadening of XRD peaks due to weakening of the crystal lattice by microstrain.

At block 206, a powder mount is prepared for each sample. The powder mount is prepared from the powder, for example, by tamping the loose powder in a slide cavity.

At block 208, XRF data is collected for each sample. At block 210, XRD data is collected for each sample.

At block 212, the weathering indices defined herein, e.g., CIA, CIW, PIA, and WIP, are constructed using molar major element oxide chemistry generated by means of the XRF data. The powder XRD data is used on the same samples as a separate technique to determine the mineralogical composition of the samples. The interpretation of the XRD patterns is used to generate weathering indices using bulk mineralogy derived calculation.

At block 214, the comparison of the weathering indices generated by the two techniques is used to develop formulas that are based on group of minerals to compute the ratios in order to model the weathering indices. The parameters for determining the weathering indices are constructed as the following equations:
Al₂O₃={(Σclays/5)}/101.96, where clays represent the sum of kaolinite, chlorite, illite, and smectite groups;
CaO*=Σcarbonates/127, where carbonate characterizes the sum of calcite, dolomite, and siderite;
Na₂O={(plagioclase+halite)/2.5}/61.9, where plagioclase is mainly represented by albite;
K₂O={(Σclays/14)}/94.19, where clays represent the sum of kaolinite, chlorite, illite, and smectite; and
MgO=dolomite.
   As described herein, all of these terms are in mole percent of the compound shown per total sample. Using these parameters, the formulas derived for the weathering indices from bulk mineralogy are as follows, using different factors, to adjust the values to match the weathering indices developed using molar major element oxide chemistry:

CIA={Al₂O₃/(Al₂O₃+CaO*+Na₂O+K₂O)}×145;

PIA={(Al₂O₃−K₂O)/((Al₂O₃−K₂O)+CaO*+Na₂O)}×180;

CIW={Al₂O₃/(Al₂O₃+CaO*+Na₂O)}×145; and

WIP={(2×Na₂O/0.35)+(MgO/0.9)+(2×K₂O/0.25)+(CaO*/0.7)}×10.

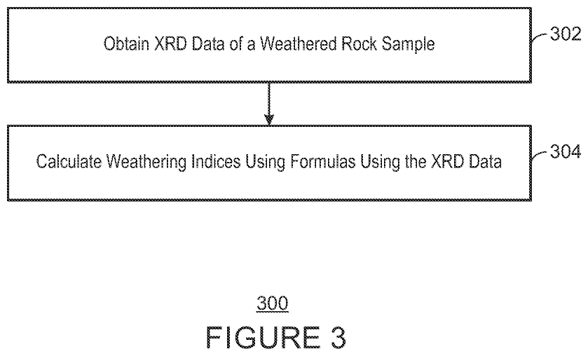

FIG. 3 is a process flow diagram of a method 300 for calculating weathering indices using formulas generated from x-ray diffraction (XRD) data. The method starts at block 302, with obtaining XRD data of weathered rock. This may be performed by taking a sample and processing as described with respect to the method 200 of FIG. 2 or by obtaining XRD data sets.

XRD data sets are widely available data in oil and gas companies. Using the techniques described herein, exploration geologists may use this data directly to model the weathering indices. This eliminates the need to request expensive and longer sample preparation-based techniques, such as using XRF and ICP, which need extensive experience in geochemistry.

XRD databases that may be used to obtain XRD data sets include the International Centre for Diffraction Data (ICDD), which is a large database of powder diffraction patterns. The XRD data sets also include the Powder Diffraction File (PDF), which includes the d-spacings (related to angle of diffraction) and the relative intensities of observable diffraction peaks. The XRD data sets were generated by manually identifying the diffraction peaks and entering them into the database for refinement.

At block 304, the XRD data sets are used to calculate the weathering indices. This is performed, for example, using the equations described with respect to the method 200 of FIG. 2.

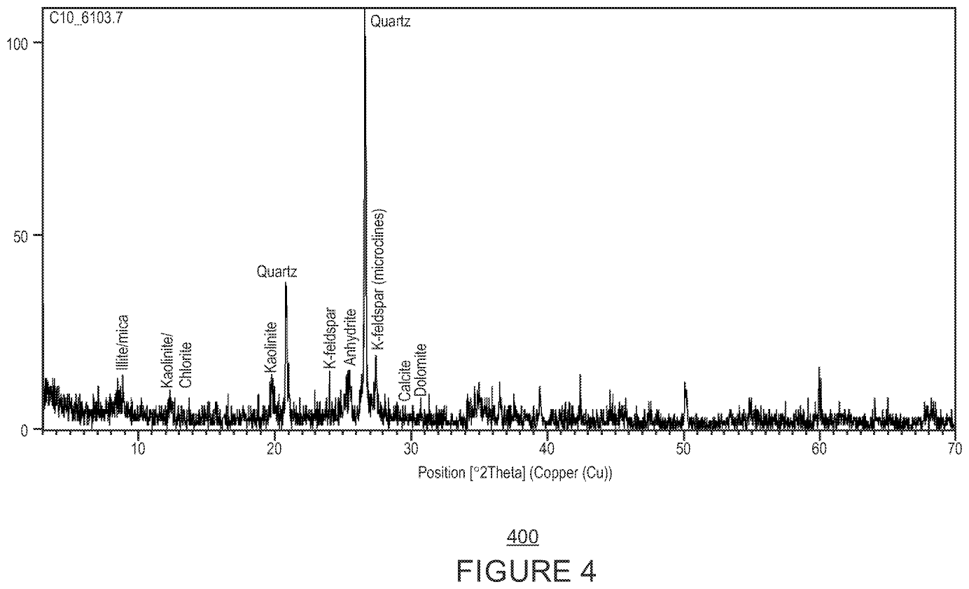

FIG. 4 is a plot 400 of a diffraction pattern of minerals used to model weathering indices. The sample was taken from the Unayzah formation of Saudi Arabia. The type locality of the Unayzah formation is in the town of Unayzah. The Unayzah formation includes sandstones, shales, and thin beds of limestone. As shown in FIG. 4, quartz is the dominant mineral composing the matrices of these rocks. However, the indices are derived from bulk mineralogy, which are used as discriminators and are based on clays, carbonate, plagioclase, halite, and dolomite. Clays represent the sum of kaolinite, chlorite, illite, and smectite, whereas carbonate characterizes the sum of calcite, dolomite, and siderite. However, plagioclase is mainly represented by albite.

The results are consistent with the observations from the XRF technique. Results indicate that powder X-ray diffraction results indicate that depth profiles of CIA, CIW, PIA, and WIP issued from mineralogy are changing according to the profiles determined by XRF, for example, the molar major element oxide chemistry.

The concentrations of Ca and Mg are primarily associated with the proportion of calcite. The distribution of Mg is linked to the presence of carbonates. High levels of Mg in conjunction with Ca are attributed to dolomite, e.g., a ⅔ ratio of Mg to Ca. Mg is also concentrated in some clay minerals such as illite. Concentrations of K and Al are linked with K feldspars, micas, and clay minerals. The concentration of Na is linked with Na-plagioclase (Albite).

### EXAMPLES

Sample Preparation

Samples of rock from the Unayzah formation, or group, were used for the studies. The samples were obtained from well core samples. Each sample was ground in a micronizing grinder to minimize microstrain that could lead to preferred orientation.

The powder samples were prepared for analysis by tamping the loose powder in the slide cavity with a razor blade having a sharp edge. This further helps to minimize preferred orientation. The same sample preparation was followed for all samples to minimize differences between samples.

XRF Measurements

Whole-rock analyses of major, trace and rare earth (REE) elements were carried out on all sampling levels using a Rigaku NEX-CG Energy Dispersive X-Ray Fluorescence (ED-XRF). The instrumental error was ±2% and ±5% for major elements and 5 ppm to 10 ppm for concentrations of trace and REE, respectively.

XRD Measurements

Mineralogical analysis was carried out on randomly oriented powders by using a Rigaku ULTIMA IV powder X-Ray diffractometer with CuKα radiation (40 kV, 40 mA), in the 3°-70° (2 θ) interval with a step size of 0.02° increment. Interpretation of the XRD patterns was made with X'Pert High Score software.

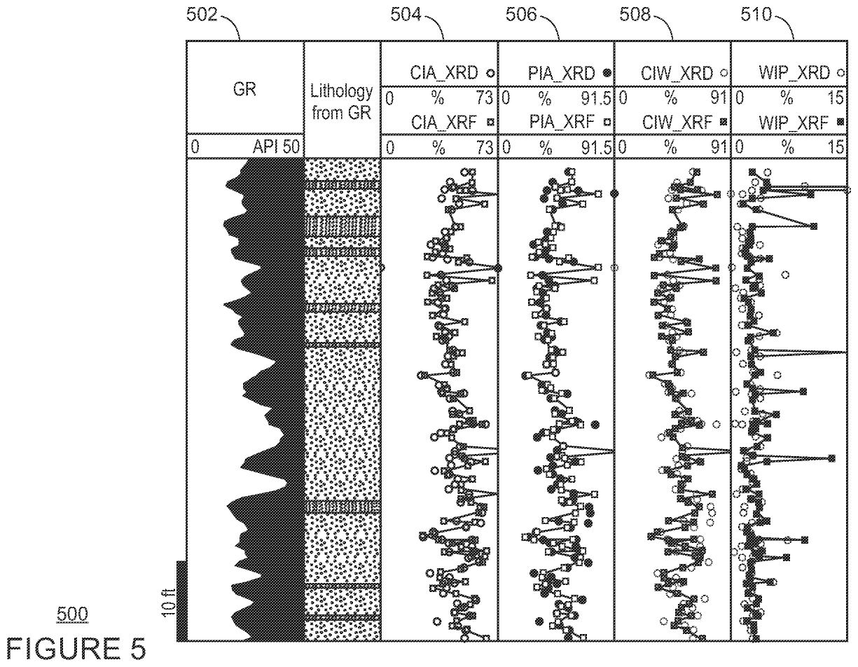

FIG. 5 is a plot 500 of depth profiles 502 comparing different indices of weathering. These include the Chemical Index of Alteration (CIA) 504, Plagioclase Index of Alteration (PIA) 506, Chemical Index of Weathering (CIW) 508, and Weathering Index of Parker (WIP) 510. These results indicate that the powder XRD results indicate that depth profiles of CIA 504, CIW 508, PIA 506, and WIP 510 issued from mineralogy are changing according to the profiles determined by XRF, e.g., the molar major element oxide chemistry.

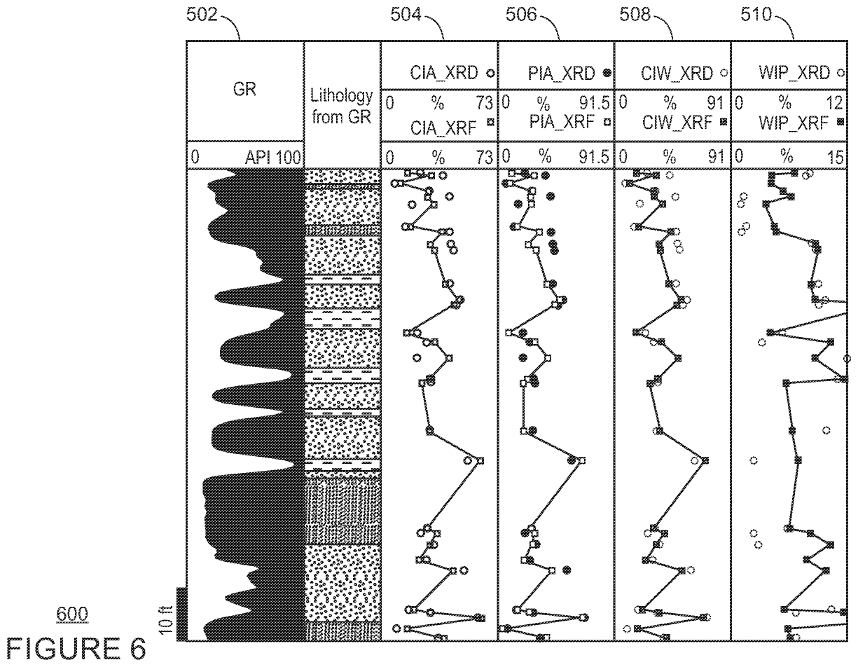

FIG. 6 is another plot 600 showing that depth profiles of the different indices are changing according to the profiles determined by XRF. Like numbered items are as described with respect to FIG. 5. As in the previous example, these results indicate that the powder XRD results indicate that depth profiles of CIA 504, CIW 508, PIA 506, and WIP 510 issued from mineralogy are changing according to the profiles determined by XRF, e.g., the molar major element oxide chemistry.

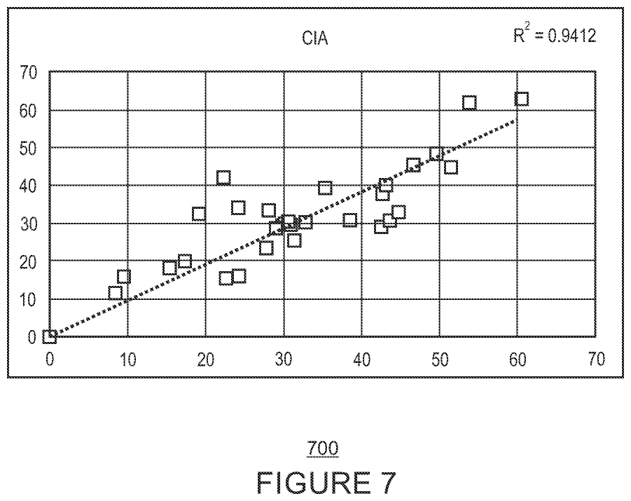

FIG. 7 is a plot 700 comparing the chemical index of alteration (CIA) determined by XRF with the CIA determined by x-ray diffraction (XRD). The comparison of the CIA determined by XRF and CIA generated by mineralogy (XRD) showed very strong correlation factor (R²=0.94).

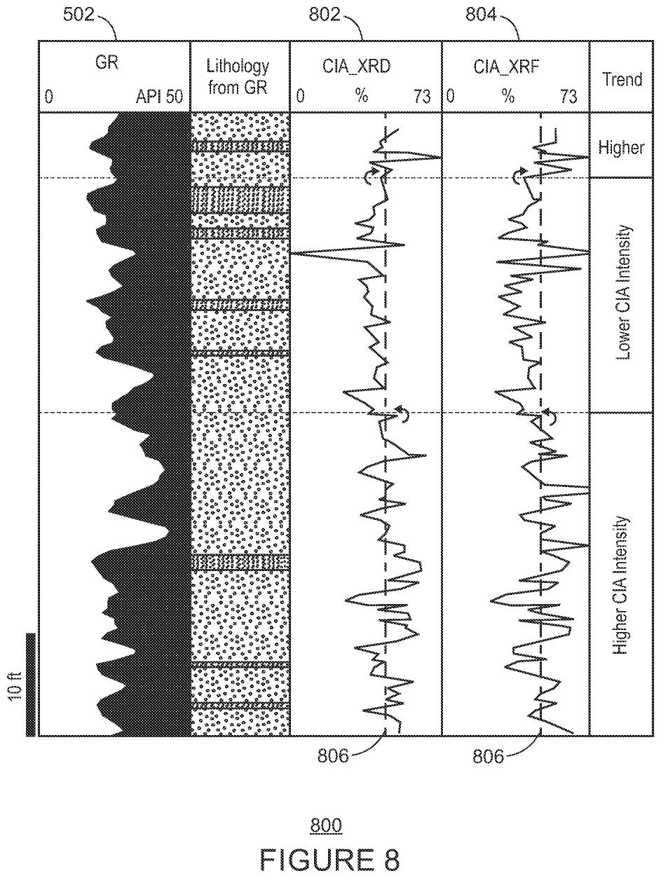

FIG. 8 is a plot 800 of depth profiles comparing the CIA determined by XRD 802 with the CIA determined by XRF 804. The plot 800 corresponds to the example of FIG. 5. Like numbered items are as discussed with respect to FIG. 5. The trend comparison of the CIA 802 determined by XRD 802 shows a strong match with the CIA determined by XRF 804. The trends are based on vertical variations of the CIA values 802 and 804 with respect to a cut-off 806.

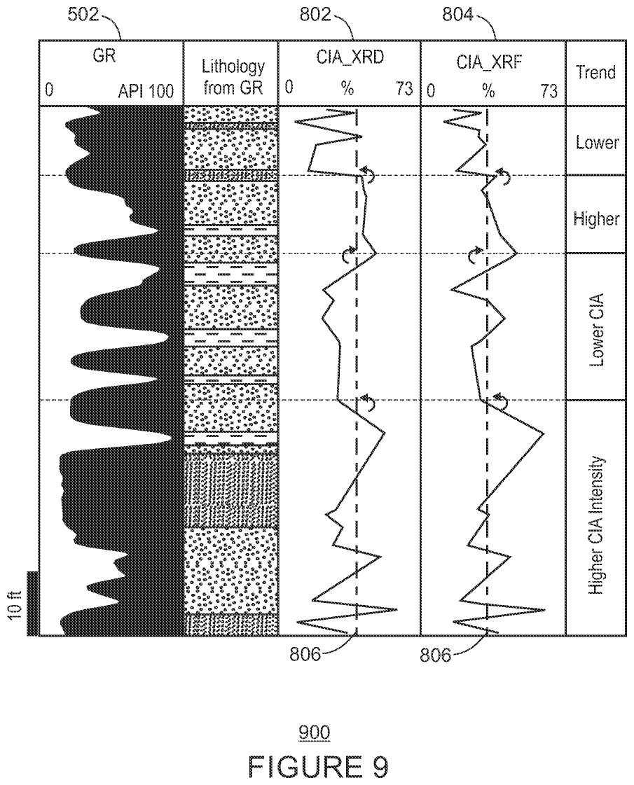

FIG. 9 is a plot 900 of depth profiles comparing trends in the CIA determined by XRD with trends determined by XRF. The plot 900 corresponds to the example of FIG. 6. Like numbered items are as discussed with respect to FIGS. 5 and 8. As in the plot 800 of FIG. 8, the trend comparison of the CIA 802 determined by XRD 802 shows a strong match with the CIA determined by XRF 804. The trends are based on vertical variations of the CIA values 802 and 804 with respect to a cut-off 806.

As shown in examples of FIGS. 5-9, the results obtained for the weathering indices using XRD are consistent with the observations from the XRF technique, indicating that using XRD to calculate the four weathering indices is comparable to using XRF.

### EMBODIMENTS

An embodiment described in examples herein provides a method for determining a weathering index using x-ray diffraction (XRD) data. The method includes obtaining XRD data of a weathered rock sample, and calculating the weathering index using a formula developed to use the XRD data.

In an aspect, the formula for the weathering index is:

CIA={Al₂O₃/(Al₂O₃+CaO*+Na₂O+K₂O)}×145.

In this formula, CIA is the chemical index of alteration, and each chemical formula represents the concentration of the named species as calculated by the following formulas:

Al₂O₃={(Σclays/5)}/101.96, where clays represent the sum of kaolinite, chlorite, illite, and smectite groups;

CaO*=Σcarbonates/127, where carbonate characterizes the sum of calcite, dolomite, and siderite;

Na₂O={(plagioclase+halite)/2.5}/61.9, where plagioclase is mainly represented by albite; and

K₂O={(Σclays/14)}/94.19, where clays represent the sum of kaolinite, chlorite, illite, and smectite.

In an aspect, the formula for the weathering index is:

PIA={(Al₂O₃−K₂O)/((Al₂O₃−K₂O)+CaO*+Na₂O)}×180.

In this formula, PIA is the plagioclase index of alteration, and each chemical formula represents the concentration of the named species as calculated by the following formulas:

Al₂O₃={(Σclays/5)}/101.96, where clays represent the sum of kaolinite, chlorite, illite, and smectite groups;

CaO*=Σcarbonates/127, where carbonate is the sum of calcite, dolomite, and siderite;

Na₂O={(plagioclase+halite)/2.5}/61.9; and

K₂O={(Σclays/14)}/94.19, where clays represent the sum of kaolinite, chlorite, illite, and smectite.

In an aspect, the formula for the weathering index is:

CIW={Al₂O₃/(Al₂O₃+CaO*+Na₂O)}×145.

In this formula, CIW is the chemical index of weathering, and each chemical formula represents the concentration of the named species as calculated by the following formulas:

Al₂O₃={(Σclays/5)}/101.96, where clays represent the sum of kaolinite, chlorite, illite, and smectite groups;

CaO*=Σcarbonates/127, where carbonate is the sum of calcite, dolomite, and siderite; and

Na₂O={(plagioclase+halite)/2.5}61.9.

In an aspect, the formula for the weathering index is:

WIP={(2×Na₂O/0.35)+(MgO/0.9)+(2×K₂O/0.25)+(CaO*/0.7)}×10.

In this formula, WIP is the weathering index of Parker, and each chemical formula represents the concentration of the named species as calculated by the following formulas:

CaO*=Σcarbonates/127, where carbonate is the sum of calcite, dolomite, and siderite;

Na₂O={(plagioclase+halite)/2.5}/61.9;

K₂O={(Σclays/14)}/94.19, where clays represent the sum of kaolinite, chlorite, illite, and smectite; and

MgO=dolomite.

In an aspect, the weathered rock sample includes a core sample from a well bore. In an aspect, the weathered rock sample includes a silt sample.

In an aspect, the XRD data is measured. In an aspect, the XRD data is obtained from an XRD database.

In an aspect, the method includes developing the formula for using the XRD data by obtaining samples of weathered rock, grinding the samples, preparing a powder mount of each sample, obtaining x-ray fluorescence (XRF) data for each sample, calculating a second weathering index using the XRF data, and constructing the weathering index using the XRD data, based, at least in part, on the second weathering index.

In an aspect, the method includes grinding the rock samples in a micronizing mill. In an aspect, the method includes packing the powder into a powder mount using a sharp edge.

Another embodiment described in examples herein provides a method for constructing a weathering index for using x-ray diffraction data by obtaining samples of weathered rock, grinding the samples, preparing a powder mount of each sample, obtaining x-ray fluorescence (XRF) data for each sample, calculating a second weathering index using the XRF data, and constructing the weathering index using the XRD data, based, at least in part, on the second weathering index.

In an aspect, the method includes grinding the rock samples in a micronizing mill. In an aspect, the method includes packing the powder into a powder mount using a sharp edge.

In an aspect, the formula for the weathering index is:

CIA={Al₂O₃/(Al₂O₃+CaO*+Na₂O+K₂O)}×145.

In this formula, CIA is the chemical index of alteration, and each chemical formula represents the concentration of the named species as calculated by the following formulas:

Al₂O₃={(Σclays/5)}/101.96, where clays represent the sum of kaolinite, chlorite, illite, and smectite groups;

CaO*=Σcarbonates/127, where carbonate characterizes the sum of calcite, dolomite, and siderite;

Na₂O={(plagioclase+halite)/2.5}/61.9, where plagioclase is mainly represented by albite; and

K₂O={(Σclays/14)}/94.19, where clays represent the sum of kaolinite, chlorite, illite, and smectite.

In an aspect, the formula for the weathering index is:

PIA={(Al₂O₃— K₂O)/((Al₂O₃−K₂O)+CaO*+Na₂O)}×180.

In this formula, PIA is the plagioclase index of alteration, and each chemical formula represents the concentration of the named species as calculated by the following formulas:

Al₂O₃={(Σclays/5)}/101.96, where clays represent the sum of kaolinite, chlorite, illite, and smectite groups;

CaO*=Σcarbonates/127, where carbonate is the sum of calcite, dolomite, and siderite;

Na₂O={(plagioclase+halite)/2.5}/61.9; and

K₂O={(Σclays/14)}/94.19, where clays represent the sum of kaolinite, chlorite, illite, and smectite.

In an aspect, the formula for the weathering index is:

CIW={Al₂O₃/(Al₂O₃+CaO*+Na₂O)}×145.

In this formula, CIW is the chemical index of weathering, and each chemical formula represents the concentration of the named species as calculated by the following formulas:

Al₂O₃={(Σclays/5)}/101.96, where clays represent the sum of kaolinite, chlorite, illite, and smectite groups;

CaO*=Σcarbonates/127, where carbonate is the sum of calcite, dolomite, and siderite; and

Na₂O={(plagioclase+halite)/2.5}61.9.

In an aspect, the formula for the weathering index is:

WIP={(2×Na₂O/0.35)+(MgO/0.9)+(2×K₂O/0.25)+(CaO*/0.7)}×10.

In this formula, WIP is the weathering index of Parker, and each chemical formula represents the concentration of the named species as calculated by the following formulas:

CaO*=Σcarbonates/127, where carbonate is the sum of calcite, dolomite, and siderite;

Na₂O={(plagioclase+halite)/2.5}/61.9;

K₂O={(Σclays/14)}/94.19, where clays represent the sum of kaolinite, chlorite, illite, and smectite; and

MgO=dolomite.

Other implementations are also within the scope of the following claims.

What is claimed is:

1. A method for determining a weathering index using x-ray diffraction (XRD) data, comprising:
obtaining XRD data of a weathered rock sample; and
calculating the weathering index using a formula developed to use the XRD data.

2. The method of claim 1, wherein the formula for the weathering index is:
CIA={Al₂O₃/(Al₂O₃+CaO*+Na₂O+K₂O)}×145,
wherein CIA is the chemical index of alteration, and each chemical formula represents the concentration of the associated species as calculated by the following formulas:
Al₂O₃={(Σclays/5)}/101.96, where clays represent the sum of kaolinite, chlorite, illite, and smectite groups;
CaO*=Σcarbonates/127, where carbonate characterizes the sum of calcite, dolomite, and siderite;
Na₂O={(plagioclase+halite)/2.5}/61.9, where plagioclase is mainly represented by albite; and
K₂O={(Σclays/14)}/94.19, where clays represent the sum of kaolinite, chlorite, illite, and smectite.

3. The method of claim 1, wherein the formula for the weathering index is:
PIA={(Al₂O₃— K₂O)/((Al₂O₃−K₂O)+CaO*+Na₂O)}×180,
wherein PIA is the plagioclase index of alteration, and each chemical formula represents the concentration of the named species as calculated by the following formulas:
Al₂O₃={(Σclays/5)}/101.96, where clays represent the sum of kaolinite, chlorite, illite, and smectite groups;
CaO*=Σcarbonates/127, where carbonate is the sum of calcite, dolomite, and siderite;
Na₂O={(plagioclase+halite)/2.5}/61.9; and
K₂O={(Σclays/14)}/94.19, where clays represent the sum of kaolinite, chlorite, illite, and smectite.

4. The method of claim 1, wherein the formula for the weathering index is:
CIW={Al₂O₃/(Al₂O₃+CaO*+Na₂O)}×145,
wherein CIW is the chemical index of weathering, and each chemical formula represents the concentration of the named species as calculated by the following formulas:
Al₂O₃={(Σclays/5)}/101.96, where clays represent the sum of kaolinite, chlorite, illite, and smectite groups;
CaO*=Σcarbonates/127, where carbonate is the sum of calcite, dolomite, and siderite; and
Na₂O={(plagioclase+halite)/2.5}/61.9.

5. The method of claim 1, wherein the formula for the weathering index is:
WIP={(2×Na₂O/0.35)+(MgO/0.9)+(2×K₂O/0.25)+(CaO*/0.7)}×10,
wherein WIP is the weathering index of Parker, and each chemical formula represents the concentration of the named species as calculated by the following formulas:
CaO*=Σcarbonates/127, where carbonate is the sum of calcite, dolomite, and siderite;
Na₂O={(plagioclase+halite)/2.5}/61.9;
K₂O={(Σclays/14)}/94.19, where clays represent the sum of kaolinite, chlorite, illite, and smectite; and
MgO=dolomite.

6. The method of claim 1, wherein the weathered rock sample comprises a core sample from a well bore.

7. The method of claim 1, wherein the weathered rock sample comprises a silt sample.

8. The method of claim 1, wherein the XRD data is measured.

9. The method of claim 1, wherein the XRD data is obtained from an XRD database.

10. The method of claim 1, comprising developing the formula for using the XRD data by:
obtaining samples of weathered rock;
grinding the samples;
preparing a powder mount of each sample;
obtaining x-ray fluorescence (XRF) data for each sample;
calculating a second weathering index using the XRF data; and
constructing the weathering index using the XRD data, based, at least in part, on the second weathering index.

11. The method of claim 10, comprising grinding the rock samples in a micronizing mill.

12. The method of claim 10, comprising packing the powder into a powder mount using a sharp edge.

13. A method for constructing a weathering index for using x-ray diffraction data by:
obtaining samples of weathered rock;
grinding the samples;
preparing a powder mount of each sample;
obtaining x-ray fluorescence (XRF) data for each sample;
calculating a second weathering index using the XRF data; and
constructing the weathering index using the XRD data, based, at least in part, on the second weathering index.

14. The method of claim 13, comprising grinding the rock samples in a micronizing mill.

15. The method of claim 13, comprising packing the powder into a powder mount using a sharp edge.

16. The method of claim 13, wherein the formula for the weathering index is:
CIA={Al₂O₃/(Al₂O₃+CaO*+Na₂O+K₂O)}×145,
wherein CIA is the chemical index of alteration, and each chemical formula represents the concentration of the named species as calculated by the following formulas:
Al₂O₃={(Σclays/5)}/101.96, where clays is the sum of kaolinite, chlorite, illite, and smectite groups;
CaO*=Σcarbonates/127, where carbonate is the sum of calcite, dolomite, and siderite;
Na₂O={(plagioclase+halite)/2.5}/61.9; and
K₂O={(Σclays/14)}/94.19, where clays is the sum of kaolinite, chlorite, illite, and smectite.

17. The method of claim 13, wherein the formula for the weathering index is:
PIA={(Al₂O₃−K₂O)/((Al₂O₃−K₂O)+CaO*+Na₂O)}×180,
wherein PIA is the plagioclase index of alteration, and each chemical formula represents the concentration of the named species as calculated by the following formulas:
Al₂O₃={(Σclays/5)}/101.96, where clays is the sum of kaolinite, chlorite, illite, and smectite groups;
CaO*=Σcarbonates/127, where carbonate is the sum of calcite, dolomite, and siderite;
Na₂O={(plagioclase+halite)/2.5}/61.9; and
K₂O={(Σcldays/14)}/94.19, where clays is the sum of kaolinite, chlorite, illite, and smectite.

18. The method of claim 13, wherein the formula for the weathering index is:
CIW={Al₂O₃/(Al₂O₃+CaO*+Na₂O)}×145,
wherein CIW is the chemical index of weathering, and each chemical formula represents the concentration of the named species as calculated by the following formulas:
Al₂O₃={(Σclays/5)}/101.96, where clays is the sum of kaolinite, chlorite, illite, and smectite groups;
CaO*=Σcarbonates/127, where carbonate is the sum of calcite, dolomite, and siderite;
Na₂O={(plagioclase+halite)/2.5}/61.9.

19. The method of claim 13, wherein the formula for the weathering index is:
WIP={(2×Na₂O/0.35)+(MgO/0.9)+(2×K₂O/0.25)+(CaO*/0.7)}×10,
wherein WIP is the weathering index of Parker, and each chemical formula represents the concentration of the named species as calculated by the following formulas:
CaO*=Σcarbonates/127, where carbonate is the sum of calcite, dolomite, and siderite;
Na₂O={(plagioclase+halite)/2.5}/61.9;
K₂O={(Σclays/14)}/94.19, where clays is the sum of kaolinite, chlorite, illite, and smectite; and
MgO=dolomite.
